(19) United States
(12) Patent Application Publication
(10) Pub. No.: US 2014/0356304 A1
(43) Pub. Date: Dec. 4, 2014

(54) Esterified Oligomeric Polyhydric Alcohols for AntiPerspirant and Deodorant Applications

(71) Applicant: BASF SE, LUDWIGSHAFEN (DE)

(72) Inventors: Yvonne Heischkel, Ludwigshafen (DE); Tatiana Drovetskaya, Martinsville, NJ (US)

(73) Assignee: BASF SE, LUDWIGSHAFEN (DE)

(21) Appl. No.: 14/372,028

(22) PCT Filed: Jan. 25, 2013
(86) PCT No.: PCT/US2013/023042

§ 371 Date: Jul. 14, 2014

(30) Foreign Application Priority Data
Jan. 26, 2012 (US) 61590871

(51) Int. Cl. A61K 8/86; A61Q 15/00

(52) U.S. Cl. CPC A61K 8/86; A61Q 15/00; A61K 2800/40
USPC 424/66; 424/65; 424/67; 424/68

(57) ABSTRACT

Antiperspirant and/or deodorant compositions commonly contain montan wax derivatives that may function as structurants preventing syneresis in cream antiperspirant and deodorant compositions, especially anhydrous cream formulations. Because montan wax derivatives are obtained via natural resources (lignite wax), the quality of the derivatives is often inconsistent and unreliable. There is therefore a need for a more reliable alternative. The present inventors have discovered that certain esterified oligomeric polyhydric alcohols, offer an effective, efficient and reliable alternative to montan wax derivatives as structuring agents.

### TECHNICAL FIELD

The present invention is directed to antiperspirant and/or deodorant compositions containing esterified oligomeric polyhydric alcohol structuring agents. These particular structuring agents provide a needed alternative to montan wax derivatives. Because montan wax derivatives are obtained via natural resources (lignite wax), the quality of the derivatives is often inconsistent and unreliable. The present applicants have determined that specific esterified oligomeric polyhydric alcohols, offer an effective, efficient and reliable alternative to montan wax derivatives as structuring agents.

### BACKGROUND OF THE INVENTION

Montan wax is a vegetable fossil wax which is part of the extractable, bituminous components of lignite and peat. Deposits of lignite, which are used for wax extraction, are found mainly in eastern Germany (Röblingen), Ukraine (Alexandrija), Russia (Baschkiren), the United States (California), and China. The composition of the extract is determined by coal quality (clay and mineral content); physical parameters such as water content, particle size, and particle-size distribution; and properties of the solvent. Furthermore, the crude extracted montan wax consists of a mixture of wax acids, wax esters, resins, asphaltenes, and dark residues. The qualitative and quantitative composition of crude montan wax is determined by the carbonized plants and extent of carbonization which is quite variable by region. Because of the crude montan wax dark color, direct use of crude montan wax is limited especially for applications such as cosmetics or personal care products. For most applications the wax must be refined and this refining process is a multistep process involving at least extractive deresinification, oxidative bleaching, and subsequent derivatization. Please see section 3, Montan Wax in Wolfmeier, U., Schmidt, H., Heinrichs, F.-L., Michalczyk, G., Payer, W., Dietsche, W., Boehlke, K., Hohner, G. and Wildgruber, J. 2000. Waxes. Ullmann's Encyclopedia of Industrial Chemistry.

Accordingly, it would therefore be advantageous to avoid the use of these montan waxes not only because of their variable source but because the multistep process for refining them is environmentally disadvantageous, especially the oxidation step which frequently requires the use of chromium acid or chromates in sulfuric acid as oxidizing agent.

Montan wax and derivatives thereof are well known as effective structurants for antiperspirants.

For example, U.S. Pat. Nos. 5,902,571, 5,718,890 and 5,891,424 disclose the use C₁₈-C₃₆ triglyceride combinations (Syncrowax® HGL-C). As the source of the mineral waxes or montan waxes vary qualitatively and quantitatively by composition, a dependable and consistent alternative is needed. Further, it would be advantageous to avoid the use of montax waxes as their refining is environmentally disadvantageous as mentioned above.

There are many types of topical antiperspirant products that are commercially available or otherwise known in the antiperspirant art. Most of these products are formulated as sprays, roll-on liquids, creams, or solid sticks, and comprise an astringent material, e.g. zirconium or aluminum salts, incorporated into a suitable topical carrier. These products are designed to provide effective perspiration and/or odor control while also being cosmetically acceptable during and after application onto the axillary area or other areas of the skin.

Soft cream antiperspirant or deodorant formulations are particularly preferred by some consumers but can be characterized by solvent syneresis in the event no effective gellant/structurant is present in the formulation. This is especially true of anhydrous systems. Although, syneresis can be minimized or eliminated in these creams by simply formulating the product into a harder, more conventional, antiperspirant stick, these antiperspirant sticks typically result in higher visible residue on the skin than soft antiperspirant creams. Accordingly, there is a need to minimize syneresis in cream antiperspirant and deodorant compositions, especially anhydrous systems by providing an alternative gellant/structurant to montan wax derivatives.

It is an object of the present disclosure to provide for an antiperspirant and/or deodorant compositions characterized by effective stability (no syneresis) and spreading performance without reliance on montan wax derivatives. It is yet another object of the present invention to provide effective stability (no syneresis) and spreading composition in antiperspirant and deodorant compositions which are essentially anhydrous creams without reliance on montan wax derivatives.

### BRIEF DESCRIPTION OF THE INVENTION

The present inventors have discovered that specific esterified oligomeric polyhydric alcohols are highly effective as alternative structurants to montan wax derivatives in antiperspirant and deodorant applications. Furthermore, it has been discovered that these esterified oligomeric polyhydric alcohols effectively prevent syneresis especially in anhydrous antiperspirant formulations while not being characterized by inconsistent quality deficiencies such as those found using the derived mineral waxes such as montan waxes.

The esterified oligomeric polyhydric alcohols are known per se, for example, in U.S. Pat. No. 4,614,604, but they are taught as lubricants for polyvinyl chloride and shaping thereof.

The present invention is therefore directed to a number of embodiments:
An antiperspirant or deodorant composition comprising
a) from about 5 to about 35 wt. %, more preferably from about 10 to about 30 wt. % and most preferably 15 to 20 wt. % antiperspirant active, wherein the wt. % is based on the total weight of the composition, and
b) oligomers formed from a minimum of 2 polyhydric aliphatic alcohol monomer units in which 30-100% of the OH groups are esterified with C₈-C₃₆ fatty acids.
A method of preparation of an antiperspirant and/or deodorant composition is envisioned by incorporating therein components a) and b) as describe above.
A method for prevention of syneresis in an antiperspirant or deodorant composition is disclosed by incorporation therein components a) and b).
Use of oligomers formed from a minimum of 2 polyhydric aliphatic alcohol monomer units in which 30-100% of the OH groups are esterified with C₈-C₃₆fatty acids as a structurant within an antiperspirant and/or deodorant, especially within an anhydrous cream antiperspirant and/or deodorant.

### DETAILED DESCRIPTION OF THE INVENTION

### Definitions

Montan Wax

Montan waxes mean vegetable fossil wax which is part of the extractable, bituminous components of lignite and peat esters of long-chain aliphatic carboxylic acids with dihydric or trihydric alcohols.

The montan wax derivatives which are typically used as structurants for cosmetics or personal care products such as antiperspirants are esters of long-chain aliphatic carboxylic acids (montan acids) with dihydric or trihydric alchols (eg. glycol or glycerol).

Comprising

Comprising for purposes of this application means that other ingredients or components may be present.

Anhydrous

“Anhydrous” as used herein means substantially free of added or free water. This means that the antiperspirant and/or deodorant compositions of the present invention may contain less than about 2%, preferably less than about 1%, more preferably less than about 0.5%, most preferably zero percent, by weight of free or added water.

Free water would not include water which is a hydrate of the antiperspirant active for example. This water is bound and thus cannot be considered free.

Synthetically Derived

Synthetically derived as used herein is a descriptor for the esterified oligomeric polyhydric alcohols used in the antiperspirant and/or deodorant applications. Thus the esterified oligomeric polyhydric alcohols differ from the montan wax derivatives in that they (the esterified oligomeric polyhydric alcohols) are formed via typical synthetic methods as opposed to extraction from natural sources such as extractable bituminous components of lignite and peat.

Percentages, Parts and Ratios

All percentages, parts and ratios are by weight of the total composition, unless otherwise specified. All such weights as they pertain to listed ingredients are based on the active level and, therefore do not include solvents or by-products that may be included in commercially available materials, unless otherwise specified.

Antiperspirant Active

The antiperspirant active, especially particulate antiperspirant active, as used herein comprises an antiperspirant active suitable for application to human skin. Suitable actives for use in the compositions are those which remain substantially unsolubilized as dispersed solid particulates in an anhydrous or substantially anhydrous system.

Antiperspirants actively reduce the amount of underarm perspiration. A deodorant by contrast reduces axillary odor through the use of an antimicrobial agent. An antiperspirant is automatically considered a deodorant because the active has at least some antimicrobial properties.

The concentration of active in the composition should be sufficient to provide the desired odor and/or wetness control.

The particulate antiperspirant active makes up about 5% to about 35 wt. %, more preferably from about 10% to about 30 wt. %, most preferably 15 to 20 wt. % based on the total weight of the antiperspirant and/or deodorant composition.

These weight percentages of the active ingredients are calculated on an anhydrous metal salt basis exclusive of water and any complexing agents such as glycine, glycine salts, or other complexing agents unless otherwise noted.

The antiperspirant active particles as formulated in the composition are in the form of dispersed solid particles having a preferred average particle size or diameter of from about 1 micron to about 100 microns, more preferably from about 1 microns to about 50 microns. 1 micron is the same as 0.001 milimeters.

The antiperspirant active for use in the antiperspirant cream compositions of the present invention include any compound, composition or other material having antiperspirant activity. Preferred antiperspirant actives include the astringent metallic salts, especially the inorganic and organic salts of aluminum, zirconium and zinc, as well as mixtures thereof. Particularly preferred are the aluminum and zirconium salts, such as aluminum halides, aluminum hydroxyhalides, zirconyl oxyhalides, zirconyl hydroxyhalides, and mixtures thereof.

Preferred aluminum salts for use in the antiperspirant cream composition include those which conform to the formula:

Al₂(OH)ₐCl_{b}x H2₂O

wherein a is from about 2 to about 5; the sum of a and b is about 6; x is from about 1 to about 6; and wherein a, b, and x may have non-integer values. Particularly preferred are the aluminum chlorhydroxides referred to as “5/6 basic chlorhydroxide”, wherein a=5, and “2/3 basic chlorhydroxide”, wherein a=4. Processes for preparing aluminum salts are disclosed in U.S. Pat. No. 3,887,692, Gilman, issued Jun. 3, 1975; U.S. Pat. No. 3,904,741, Jones et al., issued Sep. 9, 1975; U.S. Pat. No. 4,359,456, Gosling et al., issued Nov. 16, 1982; and British Patent Specification 2,048,229, Fitzgerald et al., published Dec. 10, 1980, all of which are incorporated herein by reference. Mixtures of aluminum salts are described in British Patent Specification 1,347,950, Shin et al., published Feb. 27, 1974, which description is also incorporated herein by reference.

Preferred zirconium salts for use in the antiperspirant cream composition include those which conform to the formula:

ZrO(OH)₂₋ₐClₐ. x H₂O

wherein a is from about 1.5 to about 1.87; x is from about 1 to about 7; and wherein a and x may both have non-integer values. These zirconium salts are described in Belgian Patent 825,146, Schmitz, issued Aug. 4, 1975, which description is incorporated herein by reference. Particularly preferred zirconium salts are those complexes which additionally contain aluminum and glycine, commonly known as ZAG complexes. These ZAG complexes contain aluminum chlorhydroxide and zirconyl hydroxy chloride conforming to the above described formulas. Such ZAG complexes are described in U.S. Pat. No. 3,679,068, Luedders et al., issued Feb. 12, 1974; Great Britain Patent Application 2,144,992, Callaghan et al., published Mar. 20, 1985; and U.S. Pat. No. 4,120,948, Shelton, issued Oct. 17, 1978, all of which are incorporated herein by reference.

The Structurant/Gellant or Component b)

The term “structurant” or “gellant” are used synonymously and herein mean the esterified oligomeric polyhydric alcohols as defined below.

Formula (I)

The preferred esters have a linear structure corresponding to the formula I below

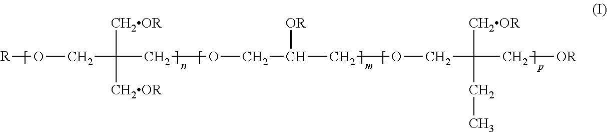

wherein R is hydrogen or the fatty acid radical

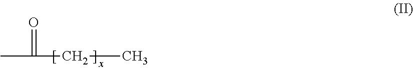

of formula (II),
x is 6-34, preferably x is 8-34, most preferably x is 14-32, n is from 0 to 10, m is from 0 to 10 and p is from 0 to 10,
with the proviso that
n+m+p≧2, preferably n+m+p≧2 and the sum does not exceed 25,
and R is 30 to 100%, preferably 50 to 90% the fatty acid acyl radical according to formula (II).

When discussing R and the percent of the fatty acid acyl radicals means the percent of OH groups which are esterified with C₈-C₃₆ fatty acids. Accordingly, the percent is not a weight percent, but a percent of the OH groups esterified.

Formula (III)

Particularly preferred structures correspond to the formula (Ill) below.

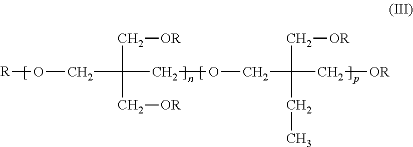

Here in formula (III) m is 0 so that the products are for example, esters of cocondensates of trimethylolpropane and pentaerythritol, n is from 2 to 8 and p is from 1 to 3.

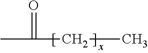

R is in this case hydrogen or the acyl radical of formula (II)
wherein x is 6-34, preferably x is 8-34 and most preferably x is 14-32;
R is 30 to 100%, preferably 50 to 90% of the fatty acid acyl radical of formula (II).

Formula (IV)

A further important esterified oligomeric polyhydric alcohols is an ester of formula (IV) defined below.

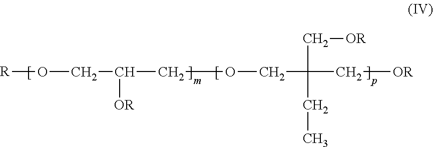

R of formula (IV) is hydrogen or the fatty acid acyl radical
of formula (II),

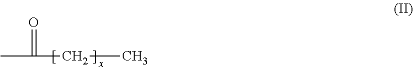

wherein x is 6-34, preferably x is 8-34, most preferably x is 14-32,
For formula (IV),
n is 0;
m is from 0 to 10 and p is from 0 to 10;
with the proviso that m+p is≧2, preferably m+p is≧2 and does not exceed 20;
R is 30 to 100%, preferably 50 to 90% the fatty acid acyl radical according to formula (II).
Formula (V)
A further important structure for the esterified oligomeric polyhydric alcohols is structure of formula (V)

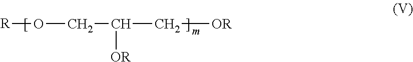

m is 3 to 10, ie the product is an oligoglycerol ester and R is hydrogen or the fatty acid acyl radical
of formula (II),

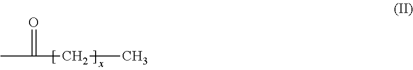

wherein x is 6-34, preferably x is 8-34, most preferably x is 14-32,
with the proviso that R is 30 to 100%, preferably 50 to 90% the fatty acid acyl radical according to formula (II).

It is important that the minimum number of monomer units in the molecule should be 2 and that the preferred maximum number of 25 units should not be exceeded. The degree of esterification is also important as defined above. It is moreover advantageous if the residual carboxylic acid concentration is below 0.4 mmol/g in the final esterified oligomeric polyhydric alcohols.

### Method of Preparation of the Esterified Oligomeric Polyhydric Alcohols

The oligomeric polyhydroxy compounds are prepared by conventional methods. Thus, for example, the polyhydroxy compounds shown in Table 1 are prepared from trimethylolpropane and glycidol (2,3-epoxypropanol), using a basic catalyst, eg. KOH, at 90-130 ° C., preferably 100.-110.° C., under a nitrogen atmosphere. In the case of products 16 and 17, a mixture of 1 mole of trimethylolpropane and 1 mole of pentaerythritol is reacted with 3.5 moles of glycidol in the presence of 0.5% by weight of KOH at 100-116° C.

The OH number is determined experimentally and from this the OH equivalent weight is calculated, which in turn can be used to calculate the amount of monobasic fatty acids required to give the desired percentage esterification.

The polyhydroxy compounds are condensed with the fatty acids, eg. stearic acid, at an elevated temperature, under vacuum or a nitrogen atmosphere, using either a metal catalyst such as dibutyl-tin maleate or dibutyl-tin dilaurate or an acid catalyst such as sulfuric acid, p-toluenesulfonic acid or methanesulfonic acid.

After condensation, the ester is usually filtered, but not bleached. However, where necessary, it can be bleached with 1-5% by weight of bleaching earth, though if the reaction is carried out carefully the products obtained mostly have a satisfactory color and do not require further bleaching. Water extraction is a further, potential purification procedure to be applied if necessary.

Please see examples 1-4 in the Examples section of the specification which describe the preparation of some of the oligomeric esters. Also, refer to U.S. Pat. No. 4,614,604 for the preparation of further examples of esterified oligomeric polyhydroxy compounds herein incorporated herein entirely by reference . See in particular Table I—esters of stearic acid and polyhydroxy compounds obtained from trimethylolpropane and/or pentaerythritol reacted with various moles of glycidol, Table 2—Esters of fatty acids and polyglycerols and Table 3—Esters of steraic acid and polycondensates of trimethylolpropane and trimethylolpropane/pentaerythritol mixtures in the stated molar ratio.

The antiperspirant and/or deodorant compositions preferably comprise from about 0.1% to about 20%, preferably about 0.5% to about 12% by weight of the gellant based on the total weight of the antiperspirant and/or deodorant.

Accordingly, an important embodiment is:

An antiperspirant and/or deodorant composition is envisioned comprising
a) from about 5 to about 35 wt. %, more preferably from about 10 to about 30 wt. % antiperspirant active, especially particulate antiperspirant active, most preferably from about 15 to about 20 wt. % wherein the wt. % is based on the total weight of the composition, and
b) defined according to formula (I),

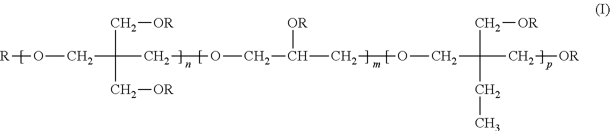

wherein R is hydrogen or the fatty acid radical

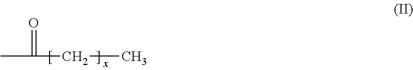

of formula (II),
x is 6-34, preferably x is 8-34, most preferably x is 14-32, n is from 0 to 10, m is from 0 to 10 and p is from 0 to 10,
with the proviso that
n+m+p≧2, preferably n+m+p≧2 and the sum does not exceed 25,
and R is 30 to 100%, preferably 50 to 90% the fatty acid acyl radical according to formula (II).

A further important embodiment is the antiperspirant and/or deodorant comprises:
a) from about 5 to about 35 wt. %, more preferably from about 10 to about 30 wt. % antiperspirant active, especially particulate antiperspirant active, most preferably from about 15 to about 20 wt. % wherein the wt. % is based on the total weight of the composition and
b) defined according to formulae (III)

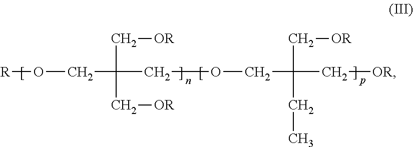

m is 0;
n is from 2 to 4 and p is from 1 to 3;
   R is in this case hydrogen or the fatty acid acyl radical of formula (II)

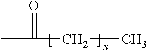

wherein x is 6-34, preferably x is 8-34 and most preferably x is 14-32;
R is 30 to 100%, preferably 50 to 90% of the fatty acid acyl radical of formula (II).
A third important embodiment is the antiperspirant and/or deodorant comprises:
a) from about 5 to about 35 wt. %, more preferably from about 10 to about 30 wt. % antiperspirant active, especially particulate antiperspirant active, most preferably from about 15 to about 20 wt. % wherein the wt. % is based on the total weight of the composition and
b) defined according to formula (IV)

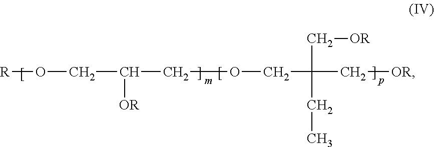

R of formula (IV) is hydrogen or the fatty acid acyl radical
of formula (II),

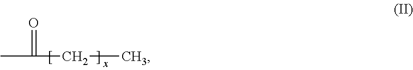

wherein x is 6-34, preferably x is 8-34, most preferably x is 14-32,
m is from 0 to 10 and p is from 0 to 10;
with the proviso that m+p is≧2, preferably m+p is≧2 and does not exceed 20;
R is 30 to 100%, preferably 50 to 90% the fatty acid acyl radical according to formula (II).
And finally,
the antiperspirant and/or deodorant comprises:
a) from about 5 to about 35 wt. %, more preferably from about 10 to about 30 wt. % antiperspirant active, especially particulate antiperspirant active, most preferably from about 15 to about 20 wt. % wherein the wt. % is based on the total weight of the composition and
b) defined by formula (V)

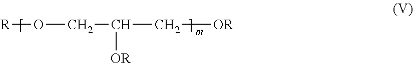

### M is 2 to 10,

R is hydrogen or the fatty acid acyl radical
of formula (II),

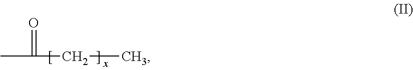

wherein x is 6-34, preferably x is 8-34, most preferably x is 14-32,
with the proviso that R is 30 to 100%, preferably 50 to 0% the fatty acid acyl radical according to formula (II).

The antiperspirant and/or deodorant compositions above contain any one or some mixture of the structurants or component b) (I, III, IV or V) in amounts ranging from about 0.1% to about 20%, preferably from about 1% to about 15%, more preferably from about 3% to about 12%, by weight of the total antiperspirant and/or deodorant composition.

Furthermore, while the antiperspirant and/or deodorant may be in any form such as a stick, solid, liquid, cream or emulsion form, the preferred form is a cream, soft stick or stick. The most preferred form is a substantially anhydrous cream. Accordingly the antiperspirant and/or deodorant may most preferably be an anhydrous cream containing substantially no free water.

Thus an important embodiment is a substantially anhydrous antiperspirant and/or deodorant cream composition comprising components a) the antiperspirant active, especially particulate antiperspirant active and b) anyone or combination of formulae (I), (III), (IV) and/or (V).

“Substantially anhydrous” as used herein means that the antiperspirant and/or deodorant compositions is substantially free of added or free water. This means that the antiperspirant and/or deodorant compositions of the present invention contain less than about 2%, preferably less than about 1%, more preferably less than about 0.5%, most preferably zero percent, by weight of free or added water.

Note that the antiperspirant may contain bound water such as explained above. For example, Al₂(OH)ₐCl_{b}.x H₂O and ZrO(OH)₂₋ₐClₐ.x H₂O normally contain bound water. The substantially anhydrous as used in the antiperspirant and/or deodorant composition does not include this bound water.

Typically the substantially anhydrous antiperspirant cream compositions of the present invention are dispersions of particulate antiperspirant solids in a continuous water-insoluble or lipophilic phase. These compositions are anhydrous systems which are suitable for use in topical cream applicators, or by other known or otherwise effective means of topically applying a cream to the skin.

Cream compositions may be characterized by viscosities ranging from 20,000 to 300,000 centipoise, preferably 40,000 to 250,000 and most preferable 50,000 to 150,000 measured by a Brookfiled Viscometer/Rheometer fitted with a T-bar type spindle. Measurements are carried out at room temperature.

Accordingly, the antiperspirant and/or deodorant cream comprises:
a) from about 5 to about 35 wt. %, more preferably from about 10 to about 30 wt. %, antiperspirant active, especially particulate antiperspirant active, most preferably from about 15 to about 20 wt. % wherein the wt. % is based on the total weight of the composition;
b) defined by any one of formulae (I), (III), (IV) or (V) described above and
c) an anhydrous liquid carrier ranging from about 10% to about 80%, preferably from about 30% to about 70%, and especially from about 45% to about 70%, by weight of the total antiperspirant and/or deodorant composition.

Anhydrous Liquid Carrier

The anhydrous antiperspirant cream compositions comprise an anhydrous liquid carrier for which serves as a carrier for the antiperspirant actives and possibly oil soluble ingredients such as vitamins, perfumes etc, wherein the anhydrous liquid carrier comprises one or more liquid carriers.

The term “liquid carrier” and “carrier” are used interchangeably herein, and refer to the anhydrous liquid carrier component of the composition, which forms a homogenous liquid with the selected structurant, component b) formulae (I), (III), (IV) or (V) as described herein.

Concentrations of the anhydrous liquid carrier in the composition will vary with the type of liquid carrier selected, depending on the actives and other ingredients in the formulation. Preferred concentrations of the anhydrous liquid carrier ranges from about 10% to about 80%, preferably from about 30% to about 70%, more preferably from about 45% to about 70%, by weight of the total antiperspirant and/or deodorant.

The anhydrous liquid carrier comprises one or more liquid carriers suitable for topical application to human skin, which carrier or combination of liquid carriers are liquid under ambient conditions. These liquid carriers may be organic or silicone-containing, volatile or nonvolatile, polar or nonpolar, provided that the carrier can form a homogenous liquid or homogenous liquid dispersion with the selected component b) at the selected structurant concentration at a temperature of from about 28° C. to about 125° C. The anhydrous liquid carrier preferably has a low viscosity to provide for improved spreading performance on the skin, more preferably less than about 50 cs (centistokes), even more preferably less than about 10 cs.

There are multiple anhydrous liquid carriers suitable for use in the antiperspirant and/or deodorant compositions comprising components a) and b) as above.

For example silicone carrier liquids are Cyclomethicone D-5 (commercially available from G. E. Silicones); Dow Corning 344, and Dow Corning 345 (commercially available from Dow Corning Corp.); and GE 7207, GE 7158 and Silicone Fluids SF-1202 and SF-1173 (available from General Electric Co.).

Examples of non-volatile, linear silicones suitable for use in the antiperspirant and deodorant compositions include Dow Corning 200, Dow Corning 225, Dow Corning 1732, Dow Corning 5732, Dow Corning 5750 (available from Dow Corning Corp.); and SF-96, SF-1066 and SF18(350) Silicone Fluids (available from G.E. Silicones).

Other suitable liquid carriers include nonpolar hydrocarbon liquids. In this context, the term “nonpolar” means that these volatile hydrocarbon liquids have a solubility parameter of less than about 7.5 (cal/cm³)^{0.5}, most typically about 5.0 (cal/cm³)^{0.5} to less than about 7.5 (cal/cm³)^{0.5}. These volatile, nonpolar hydrocarbon liquids preferably contain only hydrogen and carbon and therefore preferably contain no functional groups. Solubility parameters as described above are determined by methods well known in the chemical arts for establishing the relative polar character of a solvent or other material. A description of solubility parameters and means for determining them are described by C. D. Vaughan, “Solubility Effects in Product, Package, Penetration and Preservation” 103 Cosmetics and Toiletries 47-69, October 1988; and C. D. Vaughan, “Using Solubility Parameters in Cosmetics Formulation”, 36 J. Soc. Cosmetic Chemists 319-333, September/October, 1988, which descriptions are incorporated herein by reference.

The nonpolar, hydrocarbon liquid as a liquid carrier for use in the composition of the present invention is for example a liquid paraffin and/or isoparaffin. The nonpolar hydrocarbon liquids can have a cyclic, branched and/or chain configuration, and can be saturated or unsaturated, preferably saturated.

Specific nonlimiting examples of such hydrocarbon liquids are the isoparaffins C13-C14 Isoparaffin, C7-C8 Isoparaffin, C8-C9 Isoparaffin, C10-11 Isoparaffin, C11-C13 Isoparaffin, C11-C12 Isoparaffin, and combinations thereof. Other nonlimiting examples of suitable branched chain hydrocarbons include C12, isododecane, C16, isohexadecane, C20, isoeicosane, and combinations thereof.

Still other suitable isoparaffins include C9-C11 Isoparaffin, C9-C13 Isoparaffin, C9-C14 Isoparaffin, C10-C13 Isoparaffin, C12-C14 Isoparaffin, C13-C16 Isoparaffin, C14-C18 Isoparaffin, and hydrogenated polyisobutene.

Nonlimiting examples of other nonpolar hydrocarbon liquids suitable for use in the antiperspirant and deodorant compositions include paraffins such as dodecane, octane, decane and combinations thereof.

Yet other liquid carriers comprise branched aliphatic alcohols containing from 12 to 25 carbons, including iso-stearyl alcohol and octyldodecanol.

It will be recognized that at least some of the liquid carriers can alternatively be viewed as emollient oils. As such they can be included in order to provide both carrier and emollient functions. For example, the carrier liquid may include fatty acid and fatty alcohol esters and water insoluble ethers. Examples of such emollients include isopropyl myristate, isopropyl palmitate, cetyl acetate, cetyl propionate, di-n-butyl phthalate, diethyl sebacate, diisopropyl adipate, ethyl carbomethyl phthalate.

Polyglycol ethers are also popular choices for inclusion into the carrier as they can function as both a carrier and an emollient. The presence of the polyglycol ether imparts advantageous emollient properties, and can lower visible deposits when the composition is topically applied to human skin.

The polyglycol ether usually is derived from a low molecular weight glycol, frequently a O₂ to O₄ glycol, such as from ethylene, propylene or butylene glycol and is especially a polypropylene glycol ether. The polyglycol moiety desirably contains from 5 to 24 glycol units and in a number of preferred ethers contains from 10 to 16 glycol units, especially 10 to 16 propylene glycol units. The ether moiety is preferably aliphatic, derivable from a low molecular weight aliphatic alcohol and especially an alkanol containing up to 8 carbons, particularly 3 to 8 carbons. The alkanol is frequently propanol or butanol. For example, polypropylene glycol butyl ethers in which the polyglycol moiety contains 10 to 16 propylene glycol units, e.g. 13 or 14, are a frequent choice.

Thus the antiperspirant and/or deodorant composition, preferably an anhydrous antiperspirant and/or deodorant may further comprise an anhydrous liquid carrier selected from the group consisting of silicones, paraffins, isoparafins, branched aliphatic alcohols containing from 12 to 25 carbons, fatty acid and fatty alcohol esters, water insoluble ethers and polyglycol ethers.

The antiperspirant and/or deodorant composition, preferable anhydrous antiperspirant and/or deodorant cream or soft solid composition comprises
a) an antiperspirant, especially particulate antiperspirant,
b) an esterified oligomeric polyhydric alcohols,
c) a anhydrous liquid carrier selected from group consisting of silicone, liquid paraffin and/or isoparaffin, a fatty alcohol, branched aliphatic alcohols containing from 12 to 25 carbons, fatty acid and fatty alcohol esters, water insoluble ethers and polyglycol ethers. and
d) optionally, other additives.

### Other Optional Additives

While component b) is the primary structurant of the present antiperispirant and/or deodorant additional or secondary structurants and gellants may be included as well as other additives.

Other secondary structurants include but are not limited to fatty alcohols, ethoxylated fatty alcohols, waxes, montan wax derivatives, fatty acid esters such as mono, di or triglyceride esters (ie. glyceryl tribenhenate), dibenzylidene alditols (ie. dibenzylidene sorbitol), polyglycol ethers and amide gellants.

Suitable fatty acid esters for use as crystalline gellants include ester waxes, monoglycerides, diglycerides, triglycerides and combinations thereof. Preferred are the glyceride esters. Nonlimiting examples of suitable ester waxes including stearyl stearate, stearyl behenate, palmityl stearate, stearyl octyldodecanol, cetyl esters, cetearyl behenate, behenyl behenate, ethylene glycol distearate, ethylene glycol dipalmitate, and beeswax. Examples of commercial ester waxes include Kester waxes from Koster Keunen, Crodamol SS from Croda and Demalcare SPS from Rhone Poulenc.

The esterified fatty acid moieties may be saturated or unsaturated, substituted or unsubstituted, linear or branched, but are preferably linear, saturated, unsubstituted ester moieties derived from fatty acid materials having from about 18 to about 36 carbon atoms.

Specific examples of triglyceride gellants include, but are not limited to, tristearin, tribehenate, behenyl palmityl behenyl triglyceride, palmityl stearyl palmityl triglyceride, hydrogenated vegetable oil, hydrogenated rape seed oil, castor wax, fish oils, tripalmiten, glyceryl stearate and glyceryl distearate.

These additional gellants may be used in the composition at concentrations preferably ranging from about 0.1% to about 8%, more preferably from about 3% to about 8%, even more preferably from about 3% to about 6%, by weight of the composition.

The fatty alcohols can be saturated or unsaturated but are preferably saturated, unsubstituted, monohydric alcohols or combinations thereof. Specific examples of fatty alcohol for use in the antiperspirant and/or deodorant anhydrous cream compositions disclosed herein that are commercially available include, but are not limited to, Unilin® 550, Unilin® 700, Unilin® 425, Unilin® 400, Unilin® 350, and Unilin® 325 commercially available from Baker Petrolite.

Suitable ethoxylated fatty alcohols include, but are not limited, Unithox® 325, Unithox® 400, and Unithox® 450, Unithox® 480, Unithox® 520, Unithox® 550, Unithox® 720, Unithox® 750, all of which are available from Baker Petrolite.

Syncrowax®HGLC (CAS registry number is 91052-08-3) a montan wax derivative conforms to the formula below wherein x is 16-34

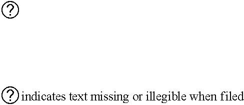

Montan wax, which is an example of mineral wax, includes glyceride esters of C18-36 carboxylic acids, hydrocarbons and other constituents.

Suitable amide gellants include monoamide gellants, diamide gellants, triamide gellants, and combinations thereof, non limiting examples of which include cocoamide MEA (monoethanolamide), stearamide, oleamide, oleamide MEA, tallow amid monoethanolamide, and the n-acyl amino acid amide derivatives may be additionally added to the present antiperspirant and/or deodorant.

Dibenzylidene alditols are for example dibenzylidene sorbitol (DBS), dibenzylidene xylitol, and dibenzylidene ribitol. The aromatic rings in each benzylidene group may be unsubstituted or substituted, as described in U.S. Pat. No. 5,200,174, which is incorporated herein by reference. When substituted, it is preferred that the benzyl ring contain an electron withdrawing group at the meta position. Typical substituted compounds include di(meta-fluorobenzylidene) sorbitol and di(meta-chlorobenzylidene) sorbitol. The preferred gelling agent is dibenzylidene sorbitol (DBS).

Accordingly, the antiperspirant and/or deodorant composition may further comprises component d) other additives wherein the other additives are selected from the group consisting of fatty alcohols, ethoxylated fatty alcohols, waxes, montan wax derivatives, fatty acid esters such as mono, di or triglyceride esters (ie. glyceryl tribehenate), dibenzylidene alditols (ie. dibenzylidene sorbitol), polyglycol ethers and amide gellants.

Especially preferred antiperspirant and/or deodorant compositions containing a further component d) are those wherein the fatty acid esters of mono, di or triglyceride esters is glyceryl tribehenate, the montan wax derivative is C₁₈-C₃₆ triglyceride and the dibenzylidene alditols is dibenzylidene sorbitol.

Wax is conventionally applied to a variety of materials and mixtures which have similar physical properties, namely they are solid at 30° C. and preferably also at 40° C.; they melt to a mobile liquid at a temperature above 30° C. but generally below 140° C. and preferably in a temperature range of 40° C. to 120° C.; they are water-insoluble and remain water-immiscible when heated above their melting point.

Waxes herein are usually selected from hydrocarbons, oxidized hydrocarbons, silicone polymers, esters of fatty acids or mixtures containing such compounds along with a minority (less than 50%) of other compounds. Naturally occurring waxes are often mixtures of compounds which include a substantial proportion likely to be a majority of fatty esters. They form crystals in the water-immiscible liquid when it cools from the heated state during processing, commonly needles or platelets.

Examples of hydrocarbon waxes include paraffin wax, microcrystalline wax and polyethylenes with molecular weight of 2,000 to 10,000. Examples of ester waxes include esters of C₁₆-C₂₂ fatty acids with glycerol or ethylene glycol and these may be made synthetically. Examples of natural waxes include beeswax, carnauba and candelilla waxes which are of vegetable origin and mineral waxes from fossil remains other than petroleum.

Suitable fatty acid gellants include, but are not limited to, 12-hydroxystearic acid and derivatives thereof, behenic acid, eurcic acid, stearic acid, C₂₀ to C₄₀ fatty acids, and related gellants. Some commercial examples of fatty acid gellants include, but are not limited to, Unicid® 400, available from BakerPetrolite.

Inorganic Thickening Agents

Inorganic thickening agents may be added to the antiperspirant and/or deodorant. Examples of inorganic thickening agents include finely divided or colloidal silicas, talc, starches, fumed silicas, and silicates, which includes montmorillonite clays and hydrophobically treated montmorillonites, e.g., bentonites, hectorites and colloidal magnesium silicates.

Polymeric Thickening Agents

Additional polymeric thickening agents (other than the structurant) may also make up the antiperspirant and/or deodorant composition.

Examples of polymeric thickening agents include polymers well known in the antiperspirant or personal care art for use in providing thickening benefits to a composition, specific examples of which include hydrogenated butylene/ethylene/styrene copolymer, polyethylene, acrylic acid polymers, ethylene acrylate copolymers, and other polymeric thickening agents described in Rheological Properties of Cosmetics and Toiletries, Edited by Dennis Laba, published by Marcel Dekker, In., New York (1993), which description is incorporated herein by reference.

The antiperspirant compositions of the present invention may further comprise one or more components which may modify the physical or chemical characteristics of the compositions or serve as additional “active” components when deposited on the skin. The compositions may also further comprise optional inert ingredients. Many such optional materials are known in the antiperspirant art and may be used in the antiperspirant compositions herein, provided that such optional materials are compatible with the essential materials described herein, or do not otherwise unduly impair product performance.

Non limiting examples of optional materials include active components such as bacteriostats, fungiostats, esterase inhibitors and skin active agents and “non-active” components such as colorants, perfumes, emulsifiers, chelants, distributing agents, preservatives, antioxidants, light stabilizers, residue masking agents, and wash-off aids.

When perspiration is present in and around the underarm region, extracellular enzymes—esterases, preferably proteases and/or lipases—which cleave esters and thus emit odor-forming substances are activated by bacteria. The esterase inhibitors are for example trialkyl citrates, such as trimethyl citrate, tripropyl citrate, tributyl citrate and, in particular, triethyl citrate inhibit the enzyme activity and thus reduce odor formation. Other substances suitable for use as esterase inhibitors are dicarboxylic acids and esters thereof such as, for example, glutaric acid, glutaric acid monoethyl ester, glutaric acid diethyl ester, adipic acid, adipic monoethyl ester, adipic acid diethyl ester, malonic acid and malonic acid diethyl ester, hydroxycarboxylic acids and esters thereof such as, for example, citric acid, malic acid, tartaric acid or tartaric acid diethyl ester.

Bactericides or bacteriostatic agents (component (d)), which influence the germ flora and kill off or inhibit the growth of perspiration-decomposing bacteria, may also be present in the formulations. Typical examples are, in particular, chitosan and phenoxyethanol. 5-Chloro-2-(2,4-dichlorophenoxy)-phenol, which is marketed by BASF SE Ludwigshaven, Germany under the name of Irgasan® (Triclosan) has also proved to be particularly effective.

Typical skin active agents are listed for examples in U.S. Pat. No. 6,403,072 and include but not limited to crystalline and non-crystalline solids such as vitamins, pharmaceuticals and other skin active materials suitable for topical application to the under arm fro the desired skin active benefit or effect. An incomplete listing for such skin actives may be found in U.S. Pat. No. 6,403,072, column 5, line 50 through column 8, line 36 herein incorporated entirely by reference.

Any fragrance material is suitable for use in the invention described herein. Fragrances suitable for use in embodiments of the invention described herein include natural products such as essential oils, flower oils, natural extracts from resins, gums, balsams, beans, mosses and other plants, and animal products such as ambergris and musk, as well as synthetic aromatic materials.

### EXAMPLES

### Example 1

### Preparation of Oligomeric Esters

1,100 g (4 moles) of stearic acid, 300 g (5 equivalents) of an adduct of trimethylolpropane with 3 moles of glycidol, having a calculated equivalent weight of 590 and a found equivalent weight of 600, and 1.4 g of dibutyl-tin maleate are condensed for 21 hours at 185.degree. C. in a 2 liter three-necked stirred flask fitted with a distillation bridge, under a nitrogen atmosphere and with stirring.
Yield: 1,326 g. Distillate: 69 g.
The product is filtered off at 90° C. on a suction filter. A clear, white product is obtained. The product has the following properties: melting point 44-46° C., iodine color number 5; OH number 49.5; acid number 11; saponification number 172.5.

### Example 2

2,310 g (8.4 moles) of stearic acid, 576 g (12 equivalents) of triglycerol (prepared from glycerol and 2 moles of glycidol) and 2.9 g of dibutyl-tin maleate are condensed for 27 hours at 188° C. in a 4 liter three-necked stirred flask equipped with a distillation bridge, under a nitrogen atmosphere and with stirring.
Yield: 2,728 g. Distillate: 150 g.
After filtration, a pale solid product is obtained.
The product has the following properties: melting point 52-53.5° C.; iodine color number 7; OH number 72.5; acid number 0.3; saponification number 175.0.

### Example 3

1,960 g (7 moles) of oleic acid (white Siegert olein), 520 g (10 equivalents) of polyglycerol prepared by self-condensation of glycerol and having an equivalent weight of 52, and 2.5 g of dibutyl-tin maleate are condensed for 20 hours at 201° C. in a 4 liter three-necked stirred flask equipped with a distillation bridge, under a nitrogen atmosphere and with stirring.
Yield: 2,345 g. Distillate 133 g.
The product is filtered through a suction filter (filter K3). A pale clear liquid is obtained.
The product has the following properties: iodine color number 9; OH number 75.0; acid number 0.3; saponification number 172.0.

### Example 4

2,200 g (8 moles) of stearic acid and 432 g (10 equivalents) of a polycondensate of trimethylolpropane and pentaerythritol in the molar ratio of 1:4 are condensed for 24 hours at 185° C. in a 4 liter three-necked stirred flask equipped with a distillation bridge, under a nitrogen atmosphere and with stirring.
Yield: 2,477 g. Distillate 137 g.
The product has the following properties: melting point 57-58° C.; iodine color number 6; OH number 47.0; acid number 0.7; saponification number 185.5.

### Example 5

862 g (3 moles) of stearic acid, 194 g of an adduct of trimethylolpropane with an average of 3 moles of glycidol, having an average molecular weight of 250 g/mol, 17.5 g (0.18 equivalents) methanesulfonic acid. in a 2 liter three-necked stirred flask fitted with a distillation bridge, under a vacuum atmosphere and with stirring.
Yield: 1,000 g. Distillate: 74 g.
The product is filtered off at 90° C. on a suction filter. A clear, white product is obtained. The product has the following properties: melting point 60-62° C., acid number 12 mg KOH/g; saponification number 168 mg KOH/g.
Formulation Prototypes

TABLE 1
Formulation of anhydrous antiperspirant cream

| Trade Name | INCI Name | |
|---|---|---|
| | | Active |
| | | ingredient, wt.¹ % |
| Reach ® AZP-908 | Aluminum Zirconium | 26.0 |
| | Tetrachlorhydrex GLY | |
| | | Inactive |
| | | Ingredients, wt. % |
| Dow Corning ® 245 | Cyclopentasiloxane | 53.5 |
| fluid | | |
| Dow Corning ® 200 | Dimethicone | 10.0 |
| Fluid | | |
| Microcrystalline Wax | Microcrystalline Wax | 2.0 |
| SP 18 | | |
| Example 5 | | 4.0 |
| Aerosil ® 200 | Silica | 1.5 |
| Pure-Dent ® B816 | Zea Mays (corn) Starch | 3.0 |

¹These weight percentages are calculated on an anhydrous metal salt basis plus water and any complexing agents such as glycine, glycine salts, or other complexing agents. The actual active percent excluding water and any complexing agents is ~20 wt. %.

The cyclomethicone and Dimethicone is heated to 73° C.-76° C. The wax sample is added followed by the microcrystalline wax with stirring. The Fumed silica is added slowly followed by the starch. All ingredients are mixed well. The batch is allowed to cool to 70° C. Temperature is maintained with addition of active ingredient (Aluminum/Zirconium Tetrachlohydrex Gly, USP). Batch cooled to 63°-65° C.

TABLE 2
Product Stability/Syneresis (RT)

| | Syneresis | Syneresis |
|---|---|---|
| Wax | 24 hrs | 1 month |
| Syncrowac ®HGLC¹ | No | No |
| No wax | Yes | Yes |
| (control) | | |
| Stearyl Alcohol | Yes | Yes |
| Commercial Product* | No | No |
| Esterified oligomeric | No | No |
| polyhydroxy alcohol | | |
| (invention)² - Example 5 | | |

¹Is C18-36 acid triglyceride (montan wax derivative). Registry no. 91052-08-3. Croda is the supplier.
*Degree Men Clinical Protection TriSolid Antiperspirant & Deodorant Solid, Cool Rush. Sold by Unilever. Ingredients listed on the package: Active Ingredients: Aluminum Zirconium Tetrachlorohydrex Gly (20%). Inactive Ingredients: Cyclopentasiloxane, Dimethicone, C18 36 Acid Triglyceride, Microcrystalline Wax, Fragrance (Parfum), Silica, Dimethicone Crosspolymer, BHT, Zea Mays (Corn) Starch.

2.C16-18 acid polyglyceride (example 5)

The wax of the invention showed good formulation compatibility and yielded smooth uniform AP/DEO cream formulations. Thus showing that the esterifed oligomeric polyhydroxyl alcohols are excellent replacements for the montan wax derivatives such as C18-36 acid triglyceride.

### Oil Spreading Test

The oil spreading test is a quantitative way of measuring the syneresis of a particular formulation.

0.1 g of the formulation is deposited in the center of the filter paper and oil release/spreading is assessed after 1, 3, 5, 10 and 30 minutes. Faster spread indicates that a formulation will be more likely to lead to syneresis ie. a less stabile formulation.

TABLE 3
Oil Spreading Test

| Wax | 1 min. | 3 min. | 5 min. | 10 min. | 30 min. |
|---|---|---|---|---|---|
| Syncrowax ® HGLC¹ | 2 | 3 | 4 | 5 | 6 |
| Blank (no wax) | 5 | 6 | 7 | 10 | 11 |
| Stearyl alcohol | 5 | 6 | 6 | 8 | 9 |
| Commercial product* | 5 | 6 | 6 | 7 | 8 |
| Esterified oligomeric | 2 | 4 | 4 | 5 | 7 |
| polyhydroxy alcohol | | | | | |
| (invention)² Example 5 | | | | | |

The above data indicates that the esterified oligomeric polyhydroxyl alcohol (invention) is an acceptable replacement for the Syncrowax ® HGLC (montan wax derivative).

### Further Formulation Prototypes

TABLE 4
Anhydrous Cream Antiperspirant

| Trade Name | INCI Name | |
|---|---|---|
| | | Active |
| | | ingredient, wt. % |
| Reach ® AZP-908 | Aluminum Zirconium | 26.0 |
| | Tetrachlorhydrex GLY | |
| | | Inactive |
| | | Ingredients, wt. % |
| Dow Corning ® 245 | Cyclopentasiloxane | 54.25 |
| fluid | | |
| Dow Corning ® 200 | Dimethicone | 10.0 |
| Fluid | | |
| Pelemol ® GTB | Glyceryl Tribehenate | 1.25 |
| Example 5 | | 4.0 |
| Aerosil ® 200 | Silica | 1.5 |
| Pure-Dent ® B816 | Zea Mays (corn) Starch | 3.0 |

TABLE 5
Antiperspirant Soft Solid

| | | |
|---|---|---|
| INCI Name | A | B |

Active
ingredient, wt. %

| | | |
|---|---|---|
| Aluminum Zirconium | 20.0 | 20.0 |
| Tetrachlorhydrex GLY | | |

Inactive
Ingredients, wt. %

| | | |
|---|---|---|
| Cyclopentasiloxane | 53.5 | 53.5 |
| Dimethicone | 10.0 | 10.0 |
| Microcrystalline Wax | 2.0 | 2.0 |
| Examples 1-5 | 2.75 | 1.25 |
| C18-36 Acid Triglyceride | 1.25 | 2.75 |
| Silica | 1.5 | 1.5 |
| Zea Mays (corn) Starch | 3.0 | 3.0 |
| Perfum | <1% | <1% |
| BHT | <1% | <1% |

### Antiperspirant Stick

TABLE 6
Antiperspirant Stick

| INCI Name | |
|---|---|
| | Active ingredient, wt. % |
| Aluminum Zirconium | 25.0 |
| Tetrachlorhydrex GLY | |
| | Inactive Ingredients, wt. % |
| Cyclopentasiloxane | 40.3 |
| Dimethicone | 6.0 |
| Stearyl alcohol | 18.00 |
| Example 5 | 4.0 |
| PEG-8 Distearate | 0.70 |
| Talc | 6.0 |

TABLE 7
Antiperspirant Soft Solids/Creams

| | Formulation | | |
|---|---|---|---|
| Ingredient | A | Formulation B | Formulation C |
| Aluminum Zirconium | 25.25 | 25.25 | 25.25 |
| Tetrachlorhydrex GLY | | | |
| Dimethicone (10 ct) | 5.0 | 5.0 | 5.0 |
| Fully hydrogenated High | 5.0 | 5.0 | 5.0 |
| Euric Acid Rapeseed oil | | | |
| Examples 1-5 | 1.25 | 1.25 | 1.25 |
| Perfume | 0.75 | 0.75 | 0.75 |
| Glycerin | 1.0 | 0.50 | 0.50 |
| Calcium Pantothenate | 0.50 | 0.50 | 3.50 |
| (solid) | | | |
| Tocopherol Acetate | 0.50 | 0 | 0 |
| Cyclopentasiloxane | QS | QS | QS |

TABLE 8
Antiperspirant Wax Sticks (Solid)

| | Formulation | Formulation | Formulation | Formulation |
|---|---|---|---|---|
| Ingredient | D | E | F | G |
| Aluminum | 20.00 | 20.00 | 20.00 | 20.00 |
| Zirconium | | | | |
| Tetra- | | | | |
| chlorhydrex | | | | |
| GLY | | | | |
| Stearyl Alcohol | 11.0 | 11.0 | 11.0 | 11.00 |
| Talc | 6.50 | 7.00 | 7.50 | 3.00 |
| Niacinamide | 3.50 | 3.50 | 0 | 7.00 |
| (solid) | | | | |
| Dimethicone | 3.00 | 5.00 | 5.00 | 5.00 |
| (50 cs) | | | | |
| Castor Wax | 2.90 | 5.00 | 5.00 | 5.00 |
| Examples 1-5 | 4.00 | 1.25 | 2.25 | 1.25 |
| Fumed Silica | 0.18 | 0.18 | 0.18 | 0.18 |
| Microthene | 0.18 | 0.18 | 0.18 | 0.18 |
| Behenyl | 0.08 | 0.08 | 0.08 | 0.08 |
| Alcohol | | | | |
| Perfume | 0.75 | 0.75 | 0.75 | 0.75 |
| Glycerin | 1.00 | 0.50 | 0.50 | 2.00 |
| Dipropylene | 0.18 | 0.18 | 0.18 | 0.18 |
| glycol | | | | |
| Calcium | 0.50 | 0.50 | 3.50 | 1.00 |
| Pantothenate | | | | |
| (solid) | | | | |
| Tocopherol | 0.50 | 0 | 0 | 0 |
| Acetate | | | | |
| Cyclopenta- | QS | QS | QS | QS |
| siloxane | | | | |

TABLE 9
Antiperspirant Low Residue Sticks (Solid)

| | Formulation | Formulation | Formulation | Formulation |
|---|---|---|---|---|
| Ingredient | H | I | J | K |
| Aluminum | 25.25 | 20.00 | 20.00 | 20.00 |
| Zirconium | | | | |
| Tetra- | | | | |
| chlorhydrex | | | | |
| GLY | | | | |
| Fully | 15.00 | 15.00 | 15.00 | 15.00 |
| hydrogenated | | | | |
| high Erucic | | | | |
| Acid | | | | |
| Rapeseed oil | | | | |
| Isopar M | 10.00 | 10.00 | 10.00 | 10.00 |
| Niacinamide | 3.50 | 3.50 | 0 | 7.00 |
| (solid) | | | | |
| Dimethicone | 5.00 | 5.00 | 5.00 | 5.00 |
| (50 cs) | | | | |
| Castor Wax | 2.90 | 5.00 | 5.00 | 5.00 |
| Examples 1-5 | 3.75 | 3.75 | 1.25 | 3.75 |
| C18-36 Acid | | | 2.50 | |
| triglyceride | | | | |
| Fumed silica | 0.18 | 0.18 | 0.18 | 0.18 |
| Perfume | 0.75 | 0.75 | 0.75 | 0.75 |
| Glycerin | 1.00 | 0.50 | 0.50 | 2.00 |
| Calcium | 0.50 | 0.50 | 3.50 | 1.00 |
| Pantothenate | | | | |
| (solid) | | | | |
| Tocopherol | 0.50 | 0 | 0 | 0 |
| Acetate | | | | |
| Triclosan | 0.30 | 0.30 | 0.30 | 0.30 |
| Cyclopenta- | QS | QS | QS | QS |
| siloxane | | | | |

1. An antiperspirant or deodorant composition comprising
a) from about 5 to about 35 wt. % a particulate antiperspirant active wherein the wt. % is based on the total weight of the composition and
b) oligomers formed from a minimum of 2 polyhydric aliphatic alcohol monomer units in which 30-100% of the OH groups are esterified with C₈-C₃₆ fatty acids.

2. The composition according to claim 1,
wherein b) is a compound of formula (I), 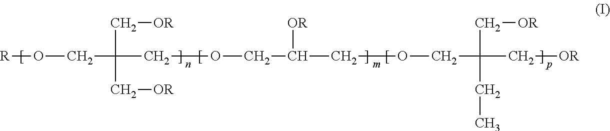
wherein R is hydrogen or the fatty acid radical 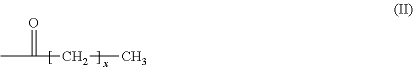
of formula (II),
x is 6-34, n is from 0 to 10, m is from 0 to 10 and p is from 0 to 10,
with the proviso that
n+m+p≧2, preferably n+m+p≧2 and the sum does not exceed 25,
and R is 30 to 100% the fatty acid acyl radical according to formula (II).

3. The composition according to claim 2,
wherein b) is a compound of formula (III), 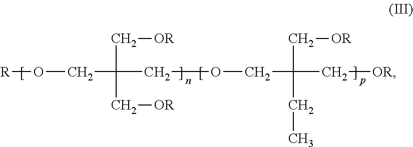
n is from 2 to 4 and p is from 1 to 3;
R is hydrogen or the fatty acid acyl radical of formula (II) 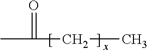
wherein x is 6-34,
R is 30 to 100%, of the fatty acid acyl radical of formula (II).

4. The composition according to claim 2,
wherein b) is a compound of formula (IV) 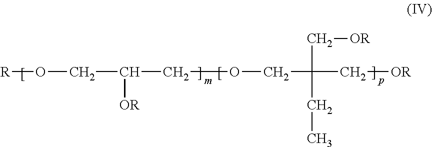
R is hydrogen or the fatty acid acyl radical
of formula (II), 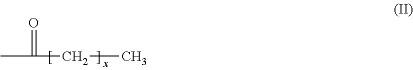
wherein x is 6-34,
m is from 0 to 10 and p is from 0 to 10;
with the proviso that m+p is≧2, and does not exceed 20;
R is 30 to 100% the fatty acid acyl radical according to formula (II).

5. The composition according to claim 1, wherein the component a) is at least one inorganic and organic salts of aluminum, zirconium and zinc, or mixtures thereof.

6. The composition according to claim 1, wherein the composition is in the form of a cream, stick, emulsion or liquid.

7. A method of preparation of an antiperspirant and/or deodorant composition by incorporating therein components a) and b) as describe in claim 1.

8. A method for prevention of syneresis in an antiperspirant or deodorant composition by incorporating therein components a) and b) as described in claim 1.

9. (canceled)
